(19) United States
(12) Patent Application Publication
(10) Pub. No.: US 2026/0077016 A1
(43) Pub. Date: Mar. 19, 2026

(54) NOVEL COMPOSITION COMPRISING ANTIBODIES

(71) Applicant: Arecor Limited, Little Chesterford (GB)

(72) Inventors: Jan JEZEK, Little Chesterford (GB); David GERRING, Little Chesterford (GB)

(21) Appl. No.: 19/395,382

(22) Filed: Nov. 20, 2025

Related U.S. Application Data
(63) Continuation of application No. 17/634,120, now abandoned.

(30) Foreign Application Priority Data
Aug. 9, 2019 (GB) 1911461.0

(51) Int. Cl. A61K 38/17; A61K 38/19; A61K 47/02; A61K 47/10; A61K 47/12; A61K 47/18; A61K 47/26; C07K 14/705; C07K 14/71; C07K 14/715

(52) U.S. Cl. CPC A61K 38/1774; A61K 38/1793; A61K 38/191; A61K 47/02; A61K 47/10; A61K 47/12; A61K 47/183; A61K 47/26; C07K 14/70532; C07K 14/71; C07K 14/7151

(57) ABSTRACT

An aqueous solution composition of pH in the range 4.0-8.5 comprising:
an antibody construct comprising an Fc domain;
optionally one or more buffers being substances having at least one ionisable group with a pKₐ in the range 3.0 to 9.5 and which pKₐ is within 2 pH units of the pH of the composition;
optionally one or more neutral amino acids; and
an uncharged tonicity modifier;
   wherein the buffers are present in the composition at a total concentration of 0-5 mM; and
   wherein the total ionic strength of the composition excluding the contribution of the engineered protein construct is less than 20 mM.

### CROSS-REFERENCE TO RELATED APPLICATIONS

This application is a continuation of U.S. patent application Ser. No. 17/634,120, filed Feb. 9, 2022, which is the U.S. national phase of International Application No. PCT/GB2020/051900 filed Aug. 7, 2020 which designated the U.S. and claims priority to GB Patent Application No. 1911461.0 filed Aug. 9, 2019, the entire contents of each of which are hereby incorporated by reference.

This invention relates to aqueous solution compositions of engineered protein constructs which comprise an Fc domain at low buffer concentrations and low ionic strength.

### BACKGROUND

Engineered proteins constructs comprising an Fc domain are widely used in therapy. The Fc domain is the C-terminal region of an antibody that interacts with cell surface receptors called Fc receptors and some proteins of the complement system and thereby activate the immune system. In IgG, IgA and IgD antibody isotypes, the Fc domain is composed of two identical protein chain fragments, each of which is derived from the second and third constant domains of the antibody's heavy chain. In IgM and IgE antibody isotypes, the Fc domain is composed of two identical protein chain fragments, each of which is derived from the second, third and fourth constant domains of the antibody's heavy chain. The molecular weight of an Fc domain may typically be in the range 25-40 kDa, and may be larger where glycosylation is present. A wide range of physiological effects result from the activation of the immune system mediated by antibody Fc domain binding, including cell lysis and degranulation of mast cells, basophils and eosinophils.

A wide range of engineered antibody protein constructs have been developed, including bispecific and trispecific antibodies. A number of engineered protein constructs have also been developed wherein the Fc, separated from the Fab parts of an antibody molecule (the parts that confer antigen binding specificity) can serve a purpose different from its physiological purpose, in particular, the purpose of extending the in vivo half-life of the protein construct.

When formulated as aqueous solutions, proteins are unstable and are susceptible to degradation and consequent loss of biological activity while stored. The degradation can be physical in nature, including aggregation, precipitation or gel formation. The degradation can also be chemical in nature, including hydrolytic cleavage, deamidation, cyclic imide formation, aspartate/glutamate isomerization or oxidation.

The rates of the degradation processes increase with increasing temperature, and protein therapeutic molecules are generally more stable at lower temperatures. However, it is often challenging to develop a therapeutic protein product that is stable in liquid form for the duration of the intended shelf-life (typically 24 months), even under refrigeration. In addition, to ensure convenience for patients there is often a need to develop products that are stable at elevated temperatures, such as up to 25° C. or up to 30° C., either for a specific period of time or for their entire shelf-life.

One of the most critical parameters to control the stability of protein therapeutics is pH. Therefore, pH optimization is a key step in formulation development. Many therapeutic proteins are formulated at a selected pH between 4.0-8.5. It is thought to be important to ensure that the pH is maintained at the selected value and pH fluctuations are minimized. Therefore, it has been understood that a certain degree of buffering capacity is needed in the formulation. Larger protein molecules typically have some self-buffering capacity due to the presence of ionisable groups amongst the amino acid side chains of the polypeptide backbone.

The present invention addresses the problem of stability of engineered protein constructs that comprise an Fc domain in aqueous solution compositions.

WO2006/138181A2 (Amgen) discloses self-buffering protein formulations which are substantially free of other buffering agents.

WO2009/073569 (Abbott) discloses aqueous formulations of antibodies such as adalimumab wherein the formulations have conductivity of less than about 2.5 mS/cm.

WO2008/084237 (Arecor) discloses protein compositions which do not comprise conventional buffers in a meaningful amount. Instead “displaced buffers” which are additives with pkₐ values at least 1 unit less than or 1 unit greater than the pH of the composition are utilised.

WO2018/094316 (Just Biotherapeutics) discloses ophthalmic formulations comprising aflibercept.

WO2013/059412 and WO2014/011629 (Coherus) disclose aqueous formulations of etanercept.

### SUMMARY OF THE INVENTION

According to the invention, there is provided an aqueous solution composition of pH in the range 4.0-8.5 comprising:
an engineered protein construct comprising an Fc domain;
optionally one or more buffers being substances having at least one ionisable group with a pKₐ in the range 3.0 to 9.5 and which pKₐ is within 2 pH units of the pH of the composition;
optionally one or more neutral amino acids; and
an uncharged tonicity modifier;
   wherein the buffers are present in the composition at a total concentration of 0-5 mM; and
   wherein the total ionic strength of the composition excluding the contribution of the engineered protein construct is less than 20 mM.

### DETAILED DESCRIPTION OF THE INVENTION

Described herein are stable aqueous solution compositions of engineered protein constructs comprising an Fc domain having absent or a low concentration of buffer and low ionic strength.

It should be noted that all references herein to “pH” refer to the pH of a composition evaluated at 25° C. All references to “pKₐ” refer to the pKₐ of an ionisable group evaluated at 25° C. (see CRC Handbook of Chemistry and Physics, 79th Edition, 1998, D. R. Lide). If required, pKₐ values of amino acid side chains as they exist in a polypeptide can be estimated using a suitable calculator.

The present inventors believe that buffers have a detrimental impact on the stability of engineered protein constructs comprising an Fc domain. Therefore, the concentration of buffer in the composition should be limited as much as possible.

The buffer(s) where present will have buffering capacity at the pH of the composition. Buffers typically comprise ionisable groups with pKₐ within 1 pH unit of the pH of the composition, however, a moiety which has ionisable groups with pKₐ 1 pH unit greater or less than the pH of the composition may also provide some buffering effect if present in a sufficient amount. In one embodiment, the (or a) buffer comprises ionisable groups with pKₐ within 1 pH unit of the pH of the composition. In another embodiment, the (or a) buffer comprises ionisable groups with pKₐ within 1.5 pH units of the pH of the composition (such as between 1 and 1.5 pH units of the pH of the composition). In a further embodiment, the (or a) buffer comprises ionisable groups with pKₐ within 2 pH units of the pH of the composition (such as between 1.5 and 2 pH units of the pH of the composition).

In an embodiment, the composition is substantially free of buffers e.g. does not contain any buffers. In an embodiment, the composition contains a single buffer. In an embodiment, the composition contains two buffers. Suitably, one or more buffers are present.

In one embodiment, the total concentration of buffers in the composition is less than 4.5 mM, such as less than 4 mM, less than 3 mM, less than 2 mM, less than 1 mM, less than 0.5 mM, less than 0.4 mM, less than 0.3 mM or less than 0.2 mM or less than 0.1 mM. In one embodiment, the total concentration of buffers is 0.1-5 mM, such as 0.5-5 mM, 0.1-4 mM, 0.5-4 mM, 0.1-3 mM, 0.5-3 mM, 0.1-2 mM, 0.5-2 mM, 0.1-1 mM or 0.5-1 mM. In one embodiment, the total concentration of buffers is 1-5 mM, 1-4 mM or 1-3 mM. In one embodiment, the aqueous solution composition is substantially free of buffer. As used herein, “substantially free” means the aqueous solution composition contains less than 0.1 mM of buffer. When considering the concentration of buffer in solution, any buffering capacity of the engineered protein construct itself should be excluded.

The pH of an aqueous solution decreases if an acid is added and increases if a base is added. At a given temperature and atmospheric pressure, the magnitude of the pH decrease on addition of an acid or the magnitude of the pH increase on addition of a base depends on (1) the amount of the acid or the base added, (2) the starting pH of the aqueous solution (i.e. prior to the addition of the acid or the base) and (3) the presence of a buffer. Thus, (1) starting from a given pH, the addition of a greater amount of an acid or a base will result in greater magnitude of pH change, (2) addition of a given amount of an acid or a base will result in the greatest pH change at neutral pH (i.e. pH 7.0) and the magnitude of the pH change will decrease as the starting pH moves away from pH 7.0 and (3) the magnitude of the pH change, starting from a given pH, will be smaller in the presence of a buffer than in the absence of a buffer. A buffer thus has the ability to reduce the change in pH if an acid or a base is added to the solution.

Suitably, a substance is considered to be a buffer if it is capable of reducing the magnitude of the pH change of a solution to 75%, preferably 50%, most preferably to 25%, compared with an identical solution that does not comprise the buffer, when either strong acid or a strong base is added resulting in 0.1 mM increase of the acid or the base in the solution.

Conversely, suitably, a substance is not considered to be a buffer if it is not capable of reducing the magnitude of the pH change of a solution to 75%, preferably 50%, most preferably to 25%, compared with an identical solution that does not comprise the substance, when either strong acid or a strong base is added resulting in 0.1 mM increase of the acid or the base in the solution.

In one embodiment, the or a buffer is an amino acid. In another embodiment, the or a buffer is not an amino acid. In an embodiment the composition is free of the amino acids lysine, arginine, histidine, glutamate and aspartate. In an embodiment the composition is free of cysteine.

Where present, suitable buffers include, but are not limited to: citrate, histidine, maleate, sulphite, glyoxylate, aspartame, glucuronate, aspartate, glutamate, tartrate, gluconate, lactate, glycolic acid, adenine, succinate, ascorbate, benzoate, phenylacetate, gallate, cytosine, p-aminobenzoic acid, sorbate, acetate, propionate, alginate, urate, 2-(N-morpholino) ethanesulphonic acid, bicarbonate, bis(2-hydroxyethyl) iminotris(hydroxymethyl) methane, N-(2-acetamido)-2-iminodiacetic acid, 2-[(2-amino-2-oxoethyl)amino]ethanesulphonic acid, piperazine, N,N′-bis(2-ethanesulphonic acid), phosphate, N,N-bis(2-hydroxyethyl)-2-aminoethanesulphonic acid, 3-[N,N-bis(2-hydroxyethyl)amino]-2-hydroxypropanesulphonic acid, triethanolamine, piperazine-N,N′-bis(2-hydroxypropanesulphonic acid), tris(hydroxymethyl)aminomethane, N-tris(hydroxymethyl)glycine and N-tris(hydroxymethyl)methyl-3-aminopropanesulphonic acid, and salts thereof, and combinations thereof. In one embodiment the buffer is selected from histidine, maleate, sulphite, glyoxylate, aspartame, glucuronate, aspartate, glutamate, tartrate, gluconate, lactate, glycolic acid, adenine, succinate, ascorbate, benzoate, phenylacetate, gallate, cytosine, p-aminobenzoic acid, sorbate, acetate, propionate, alginate, urate, 2-(N-morpholino) ethanesulphonic acid, bicarbonate, bis(2-hydroxyethyl) iminotris(hydroxymethyl) methane, N-(2-acetamido)-2-iminodiacetic acid, 2-[(2-amino-2-oxoethyl)amino]ethanesulphonic acid, piperazine, N,N′-bis(2-ethanesulphonic acid), phosphate, N,N-bis(2-hydroxyethyl)-2-aminoethanesulphonic acid, 3-[N, N-bis(2-hydroxyethyl)amino]-2-hydroxypropanesulphonic acid, triethanolamine, piperazine-N,N′-bis(2-hydroxypropanesulphonic acid), tris(hydroxymethyl)aminomethane, N-tris(hydroxymethyl)glycine and N-tris(hydroxymethyl)methyl-3-aminopropanesulphonic acid, and salts thereof, and combinations thereof. In one embodiment, the buffer is selected from citrate, maleate, sulphite, glyoxylate, aspartame, glucuronate, tartrate, gluconate, lactate, glycolic acid, adenine, succinate, ascorbate, benzoate, phenylacetate, gallate, cytosine, p-aminobenzoic acid, sorbate, acetate, propionate, alginate, urate, 2-(N-morpholino) ethanesulphonic acid, bicarbonate, bis(2-hydroxyethyl) iminotris(hydroxymethyl) methane, N-(2-acetamido)-2-iminodiacetic acid, 2-[(2-amino-2-oxoethyl)amino]ethanesulphonic acid, piperazine, N,N′-bis(2-ethanesulphonic acid), phosphate, N,N-bis(2-hydroxyethyl)-2-aminoethanesulphonic acid, 3-[N,N-bis(2-hydroxyethyl)amino]-2-hydroxypropanesulphonic acid, triethanolamine, piperazine-N,N′-bis(2-hydroxypropanesulphonic acid), tris(hydroxymethyl)aminomethane (TRIS), N-tris(hydroxymethyl)glycine and N-tris(hydroxymethyl)methyl-3-aminopropanesulphonic acid, and salts thereof, and combinations thereof. In one embodiment, the buffer is selected from the group consisting of citrate, histidine, maleate, tartrate, lactate, benzoate, acetate, bicarbonate, phosphate and tris(hydroxymethyl)aminomethane (TRIS), such as selected from the group consisting of histidine, maleate, tartrate, lactate, benzoate, acetate, bicarbonate, phosphate and tris(hydroxymethyl)aminomethane (TRIS), in particular histidine, lactate, acetate, phosphate and tris(hydroxymethyl)aminomethane (TRIS), for example, the buffer is phosphate or tris(hydroxymethyl)aminomethane (TRIS). Alternatively, the buffer is phosphate or citrate.

In an embodiment, the composition does not comprise sodium phosphate. In an embodiment, if the composition comprises phosphate buffer (e.g. sodium phosphate) suitably the concentration is less than 4.5 mM e.g. less than 4.0 mM.

The principal solvent for compositions of the invention is water, such as water for injection. Other components of the compositions (e.g. a polyol) may contribute to solubilisation of the engineered protein construct.

The composition comprises an uncharged tonicity modifier, such as a polyol. Examples of uncharged tonicity modifiers include glycerol, 1,2-propanediol, mannitol, sorbitol, sucrose, trehalose, PEG300 and PEG400. Suitably the uncharged tonicity modifier is selected from glycerol, mannitol, 1,2-propanediol and sucrose. When included, an uncharged tonicity modifier is typically employed in the composition at a concentration of 50-1000 mM, for example 200-500 mM, such as about 300 mM.

The composition suitably has an osmolarity which is physiologically acceptable and thus suitable for parenteral administration. Thus, the osmolarity of the composition is suitably 200-500 mOsm/L e.g. about 300 mOsm/L. The composition is, for example, isotonic with human plasma. Compositions may also be hypotonic, or hypertonic, e.g. those intended for dilution prior to administration.

The composition may optionally comprise a neutral amino acid. As used herein, a neutral amino acid is an amino acid the side chain of which does not contain an ionisable group which is significantly ionised (e.g. more than 20% especially more than 50% of the side chain have a minus or plus charge) at the pH of the composition. Example neutral amino acids are selected from glycine, methionine, proline, alanine, valine, leucine, isoleucine, phenylalanine, tyrosine, tryptophan, serine, threonine, asparagine and glutamine and in particular the L isomers thereof.

Suitably, neutral amino acids are selected from glycine, methionine, proline and alanine, in particular are selected from proline and glycine especially proline.

The total concentration of the one or more neutral amino acids when present may for example be 20-250 mM e.g. 20 to 200 mM e.g. 50-150 mM e.g. 50-100 mM or 25-75 mM. Alternatively it may be 100-250 mM e.g. 150-200 mM.

The present inventors believe that the presence of ions has a detrimental impact on the stability of engineered protein constructs comprising an Fc domain. Therefore, the ionic strength of the composition should be limited as much as possible.

The total ionic strength of the composition excluding the contribution of the engineered protein construct is less than 20 mM suitably less than 10 mM e.g. less than 5 mM. The term “total ionic strength” is used herein as the following function of the concentration of all ions in a solution:

$$I = {\sum }_{x = 1}^{n} {c}_{x} {z}_{x}^{2} / 2$$

where cₓ is molar concentration of ion x (mol L⁻³), zₓ is the net charge of ion cₓ. The sum covers all ions (n) present in the solution excluding the contribution of the engineered protein construct. It will be understood that optional neutral amino acids have a net charge of zero in the compositions of the invention and do not thus contribute to the total ionic strength. In any event, the contribution of any neutral amino acids is not included.

The pH of the composition is between 4.0 and 8.5, such as between 4.0 and 7.5 or between 5.0 and 8.5 e.g between 6.0 and 8.5 e.g. between 6.5 and 8.5 or between 6.0 and 7.5, such as between 7.0 and 7.5. Other ranges of interest include between 5.0 and 8.0 e.g. between 5.0 and 7.5 e.g. between 5.5 and 7.5 especially between 6.0 and 7.5.

The compositions of the invention comprise an engineered protein construct. Engineered protein constructs are non-natural proteins typically made as a product of generic engineering or synthetic chemistry. Fully human antibodies (and the natural antibodies of other non-human species), even when produced by expression in a heterologous host, such as a bacterium or fungus, are not embraced by the term “engineered protein construct”. Human antibodies produced by non-human animals (such as mice) engineered to have a human immune system are also not embraced by the term “engineered protein construct”. For example, adalimumab is not an engineered protein construct. Engineered protein constructs do include chimeric antibodies and humanized antibodies.

Engineered protein constructs of the invention comprise an Fc domain. An Fc domain is the domain of an antibody that interacts with an Fc receptor or some proteins of the complement system to activate the immune system and includes derivatives thereof. Fc domains may be derived from IgG (e.g. IgG1, IgG2, IgG3 or IgG4), IgA (e.g. IgA1 or IgA2), IgD, IgM, IgY and IgE isotypes for example. Fc domains derived from IgG, IgA and IgD isotypes comprise two identical protein chain fragments connected by disulfide bonds each of which is derived from the second and third constant domains of the antibody's heavy chain. Fc domains derived from IgM and IgE isotypes comprise three identical protein chain fragments connected by disulfide bonds each of which is derived from the second, third and fourth constant domains of the antibody's heavy chain. Fc domains may optionally be glycosylated. Most suitably the Fc domain is the Fc domain of an IgG, especially IgG1. Fc domains may typically have a molecular weight of 25-40 kDa which may be higher in the case of glycosylated Fc domains. Derivatives of Fc domains which are embraced by the term include domains known as Fcabs in which the Fc domain is modified to include an antigen binding site (see Protein Engineering, Design and Selection (2017) 30 (9) 657-671). Further examples of derivatives include conjugated derivatives e.g. such as engineered protein constructs comprising an Fc domain conjugated to another moiety. Such moieties include chemically inert polymers such as PEG. In some embodiments, the Fc domain contains one or more modifications that alters one or more properties of the engineered protein construct, such as serum half-life, complement fixation, Fc receptor binding, and/or effector function (e.g. antigen-dependent cellular cytotoxicity).

In an embodiment, the engineered protein construct is a fusion of an Fc domain with a heterologous polypeptide. One and preferably both chains of the Fc domain are fused to a heterologous polypeptide. A heterologous polypeptide is a polypeptide that is not naturally found in a contiguous sequence with the Fc domain or a chain thereof and in particular is not the antigen binding part of an antibody (i.e. the Fab part). Suitably each chain of the Fc domain is fused to the same heterologous polypeptide such that the engineered protein construct is homodimeric.

In an embodiment, the heterologous polypeptide is capable of binding a ligand, preferably a specific ligand. The heterologous polypeptide may be capable of interacting with another protein, for example, a protein that has a role in the human body (such as, without limitation, a cytokine). In an embodiment, the heterologous polypeptide is selected from cytokines, growth factors, blood clotting factors, enzymes, receptor proteins, GLP-1 agonists and functional fragments and domains thereof.

In an embodiment, the heterologous polypeptide is capable of binding to tumour necrosis factor (TNF) e.g. TNFα, and for example may comprise a TNF receptor, e.g. TNF receptor 2, especially a soluble form thereof. In an embodiment, the heterologous polypeptide is capable of binding to CD80 or CD86 and, for example, comprises the extracellular domain of CLTA-4 or a portion thereof. In an embodiment, the heterologous polypeptide is capable of binding to VEGF and for example comprises the extracellular domain of VEGFR1 and/or VEGR2 or a portion thereof. In an embodiment, the heterologous polypeptide is capable of binding to IL-1 and for example comprises the extracellular domain of IL-1R11 and/or IL-1RAcP or a portion thereof. In an embodiment, the heterologous polypeptide is capable of binding to a thrombopoietin receptor such as c-Mpl. In an embodiment, the heterologous polypeptide is a blood clotting factor such as Factor VIII or Factor IX or a portion thereof. In an embodiment, the heterologous polypeptide is a hActRIIb protein or a derivative thereof. In an embodiment the heterologous polypeptide is a protease inhibitor. In an embodiment the heterologous polypeptide is a GLP-1 agonist.

Exemplary engineered protein constructs which contain an Fc domain include etanercept, abatacept, belatacept, aflibercept, rilonacept, romiplostim, eloctate, luspatercept, dulaglutide and alprolix. In one embodiment the engineered protein construct is dulaglutide. In one embodiment the engineered protein construct is abatacept. In one embodiment the engineered protein construct is aflibercept. In one embodiment the engineered protein construct is etanercept.

In an embodiment, the engineered protein construct does comprise an antigen binding part of an antibody (i.e. an Fab part). In an embodiment, the engineered protein construct is a bispecific antibody in the format of a 4-chain antibody having two different variable binding regions. In an embodiment, the engineered protein construct is a bispecific antibody in the format of a 2-chain antibody (i.e. a heavy chain only antibody) having two different variable binding regions. Heavy chain only antibodies can, for example, be derived from antibodies isolated from Camelids. Whether in 2-chain or 4-chain format, such a bispecific antibody may, for example, have a pair of 3 CDRs for arms a and b of the antibody denoted CDR1a, CDR2a, CDR3a, CDR1b, CDR2b and CDR3b wherein CDR1a is not the same as CDR1b and/or CDR2a is not the same as CDR2b and/or CDR3a is not the same as CDR3b. Suitably none of the 6 CDRs is the same as any other of the 6 CDRs.

In an embodiment, the engineered protein construct is a bispecific antibody or a trispecific antibody in the format of an Fcab in which the Fc domain has been modified to include an antigen binding site.

In certain embodiments, two engineered protein constructs can associate e.g. via disulfide bonds to form a dimeric protein.

The engineered protein construct is preferably a therapeutic engineered protein construct. Such an engineered protein construct has a desirable therapeutic or prophylactic activity and is indicated for the treatment, inhibition or prevention of a disease or medical disorder. In certain embodiments, the engineered protein construct is substantially pure, that is, the composition comprises a single engineered protein construct and no substantial amount of any additional protein. In preferred embodiments, the engineered protein construct comprises at least 99%, preferably at least 99.5% and more preferably at least about 99.9% of the total protein content of the composition. In preferred embodiments the engineered protein construct is sufficiently pure for use in a pharmaceutical composition.

The engineered protein construct is suitably present in the composition at a concentration of about 1-400 mg/ml, suitably 10-200 mg/ml, more suitably 20-100 mg/ml e.g. about 50 mg/ml.

The composition may comprise a non-ionic surfactant. The non-ionic surfactant may for example be selected from the group consisting of a polysorbate, an alkyl glycoside, an alkyl ether of polyethylene glycol, a block copolymer of polyethylene glycol and polypropylene glycol, and an alkylphenyl ether of polyethylene glycol.

A particularly suitable class of non-ionic surfactants is the polysorbates (fatty acid esters of ethoxylated sorbitan), such as polysorbate 20 or polysorbate 80. Polysorbate 20 is a mono ester formed from lauric acid and polyoxyethylene (20) sorbitan in which the number 20 indicates the number of oxyethylene groups in the molecule. Polysorbate 80 is a mono ester formed from oleic acid and polyoxyethylene (20) sorbitan in which the number 20 indicates the number of oxyethylene groups in the molecule. Polysorbate 20 is known under a range of brand names including in particular Tween 20, and also Alkest TW 20. Polysorbate 80 is known under a range of brand names including in particular Tween 80, and also Alkest TW 80. Other suitable polysorbates include polysorbate 40 and polysorbate 60.

Another suitable class of non-ionic surfactants is the alkyl glycosides, especially dodecyl maltoside. Other alkyl glycosides include dodecyl glucoside, octyl glucoside, octyl maltoside, decyl glucoside, decyl maltoside, tridecyl glucoside, tridecyl maltoside, tetradecyl glucoside, tetradecyl maltoside, hexadecyl glucoside, hexadecyl maltoside, sucrose monooctanoate, sucrose mono decanoate, sucrose monododecanoate, sucrose monotridecanoate, sucrose monotetradecanoate and sucrose monohexadecanoate.

Another suitable class of non-ionic surfactants is alkyl ethers of polyethylene glycol, especially those known under a brand name Brij, such as selected from polyethylene glycol (2) hexadecyl ether (Brij 52), polyethylene glycol (2) oleyl ether (Brij 93) and polyethylene glycol (2) dodecyl ether (Brij L4). Other suitable Brij surfactants include polyethylene glycol (4) lauryl ether (Brij 30), polyethylene glycol (10) lauryl ether (Brij 35), polyethylene glycol (20) hexadecyl ether (Brij 58) and polyethylene glycol (10) stearyl ether (Brij 78).

Another suitable class of non-ionic surfactants is block copolymers of polyethylene glycol and polypropylene glycol, also known as poloxamers, especially poloxamer 188, poloxamer 407, poloxamer 171 and poloxamer 185. Poloxamers are also known under brand names Pluronics or Koliphors. For example, poloxamer 188 is marketed as Pluronic F-68.

Another suitable class of non-ionic surfactants are alkylphenyl ethers of polyethylene glycol, especially 4-(1,1,3,3-tetramethylbutyl)phenyl-polyethylene glycol, also known under a brand name Triton X-100.

In one embodiment, the non-ionic surfactant is a polysorbate or a poloxamer, and is suitably a polysorbate. The concentration of the non-ionic surfactant in the composition will typically be in the range 10-2000 μg/ml, such as 50-1000 μg/ml, 100-500 μg/ml or about 200 μg/ml.

The compositions of the invention may additionally comprise a preservative such as a phenolic or a benzylic preservative. The preservative is suitably selected from the group consisting of phenol, m-cresol, chlorocresol, benzyl alcohol, propyl paraben and methyl paraben, in particular phenol, m-cresol and benzyl alcohol. The concentration of preservative is typically 10-100 mM, for example 20-80 mM, such as 25-50 mM. The optimal concentration of the preservative in the composition is selected to ensure the composition passes the Pharmacopoeia Antimicrobial Effectiveness Test (USP <51>, Vol. 32).

In one embodiment, the invention provides an aqueous solution composition of pH in the range 4.0-8.5 comprising:
an engineered protein construct comprising an Fc domain;
optionally one or more buffers being substances having at least one ionisable group with a pKₐ in the range 3.0 to 9.5 and which pKₐ is within 2 pH units of the pH of the composition;
one or more neutral amino acids; and
an uncharged tonicity modifier;
   wherein the buffers are present in the composition at a total concentration of 0-5 mM; and
   wherein the total ionic strength of the composition excluding the contribution of the engineered protein construct is less than 20 mM. In one specific sub-embodiment, one or more buffers are present in said formulation at a total concentration in the composition of 0.1-5 mM such as 1-3 mM.

In one embodiment, the invention provides an aqueous solution composition of pH in the range 6.0-8.5 comprising:
an engineered protein construct comprising an Fc domain;
optionally one or more buffers being substances having at least one ionisable group with a pKₐ in the range 4.0 to 9.5 e.g. 5.0 to 9.5 and which pKₐ is within 2 pH units e.g. within 1.5 pH units e.g. within 1 pH unit of the pH of the composition;
optionally one or more neutral amino acids; and
an uncharged tonicity modifier;
   wherein the buffers are present in the composition at a total concentration of 0-5 mM; and
   wherein the total ionic strength of the composition excluding the contribution of the engineered protein construct is less than 20 mM. In one specific sub-embodiment, one or more buffers are present in said formulation at a total concentration in the composition of 0.1-5 mM such as 1-3 mM.

In one embodiment, the invention provides an aqueous solution composition of pH in the range 4.0-8.5 comprising:
an engineered protein construct comprising an Fc domain which is a bispecific antibody in the format of a 2-chain antibody having two different variable binding regions;
optionally one or more buffers being substances having at least one ionisable group with a pKₐ in the range 3.0 to 9.5 and which pKₐ is within 2 pH units e.g. within 1.5 pH units e.g. within 1 pH unit of the pH of the composition;
optionally one or more neutral amino acids; and
an uncharged tonicity modifier e.g. a polyol;
   wherein the buffers are present in the composition at a total concentration of 0-5 mM; and
   wherein the total ionic strength of the composition excluding the contribution of the engineered protein construct is less than 20 mM. In one specific sub-embodiment, one or more buffers are present in said formulation at a total concentration in the composition of 0.1-5 mM such as 1-3 mM.

In one embodiment, the invention provides an aqueous solution composition of pH in the range 4.0-8.5 comprising:
an engineered protein construct comprising an Fc domain;
optionally one or more buffers being substances having at least one ionisable group with a pKₐ in the range 3.0 to 9.5 and which pKₐ is within 2 pH units of the pH of the composition;
one or more neutral amino acids;
an uncharged tonicity modifier e.g. a polyol; and
a non-ionic surfactant;
   wherein the buffers are present in the composition at a total concentration of 0-5 mM; and
   wherein the total ionic strength of the composition excluding the contribution of the engineered protein construct is less than 20 mM. In one specific sub-embodiment, one or more buffers are present in said formulation at a total concentration in the composition of 0.1-5 mM such as 1-3 mM.

In one embodiment, the invention provides an aqueous solution composition of pH in the range 4.0-8.5 comprising:
an engineered protein construct comprising an Fc domain;
optionally one or more buffers being substances having at least one ionisable group with a pKₐ in the range 3.0 to 9.5 and which pKₐ is within 2 pH units of the pH of the composition;
one or more neutral amino acids e.g. selected from glycine, methionine, proline and alanine;
an uncharged tonicity modifier e.g. a polyol; and
a preservative
   wherein the buffers are present in the composition at a total concentration of 0-5 mM; and
   wherein the total ionic strength of the composition excluding the contribution of the engineered protein construct is less than 20 mM. In one specific sub-embodiment, one or more buffers are present in said formulation at a total concentration in the composition of 0.1-5 mM such as 1-3 mM.

In one embodiment, the invention provides an aqueous solution composition of pH in the range 4.0-8.5 comprising:
an engineered protein construct comprising an Fc domain which is a fusion of an Fc domain with a heterologous protein selected from cytokines, growth factors, blood clotting factors, enzymes, receptor proteins, GLP-1 agonists and functional fragments and domains thereof;
optionally one or more buffers being substances having at least one ionisable group with a pKₐ in the range 3.0 to 9.5 and which pKₐ is within 2 pH units of the pH of the composition;
optionally one or more neutral amino acids; and
an uncharged tonicity modifier e.g. a polyol;
   wherein the buffers are present in the composition at a total concentration of 0-5 mM; and
   wherein the total ionic strength of the composition excluding the contribution of the engineered protein construct is less than 20 mM. In one specific sub-embodiment, one or more buffers are present in said formulation at a total concentration in the composition of 0.1-5 mM such as 1-3 mM.

In one embodiment, the invention provides an aqueous solution composition of pH in the range 4.0-8.5 comprising:
an engineered protein construct comprising an Fc domain which is a bispecific antibody in the format of a 4-chain antibody having two different variable binding regions;
optionally one or more buffers being substances having at least one ionisable group with a pKₐ in the range 3.0 to 9.5 and which pKₐ is within 2 pH units of the pH of the composition;
optionally one or more neutral amino acids; and
an uncharged tonicity modifier e.g. a polyol;
   wherein the buffers are present in the composition at a total concentration of 0-5 mM; and
   wherein the total ionic strength of the composition excluding the contribution of the engineered protein construct is less than 20 mM. In one specific sub-embodiment, one or more buffers are present in said formulation at a total concentration in the composition of 0.1-5 mM such as 1-3 mM.

In one embodiment, the invention provides an aqueous solution composition of pH in the range 4.0-8.5 comprising:
an engineered protein construct comprising an Fc domain which is a bispecific antibody in the format of a 2-chain antibody having two different variable binding regions;
optionally one or more buffers being substances having at least one ionisable group with a pKₐ in the range 3.0 to 9.5 and which pKₐ is within 2 pH units of the pH of the composition;
optionally one or more neutral amino acids; and
an uncharged tonicity modifier e.g. a polyol;
   wherein the buffers are present in the composition at a total concentration of 0-5 mM; and
   wherein the total ionic strength of the composition excluding the contribution of the engineered protein construct is less than 20 mM. In one specific sub-embodiment, one or more buffers are present in said formulation at a total concentration in the composition of 0.1-5 mM such as 1-3 mM.

In one embodiment, the invention provides an aqueous solution composition of pH in the range 6.0-7.5 comprising:
an engineered protein construct comprising an Fc domain which is a bispecific antibody in the format of a 2-chain antibody having two different variable binding regions;
optionally one or more buffers being substances having at least one ionisable group with a pKₐ in the range 5.0 to 8.5 and which pKₐ is within 2 pH units e.g. within 1.5 pH units e.g. within 1 pH unit of the pH of the composition;
optionally one or more neutral amino acids; and
an uncharged tonicity modifier e.g. a polyol;
   wherein the buffers are present in the composition at a total concentration of 0-5 mM; and
   wherein the total ionic strength of the composition excluding the contribution of the engineered protein construct is less than 20 mM. In one specific sub-embodiment, one or more buffers are present in said formulation at a total concentration in the composition of 0.1-5 mM such as 1-3 mM.

In one embodiment, the invention provides an aqueous solution composition of pH in the range. 6.0 to 8.5 e.g. 6.5 to 8.5 comprising:
an engineered protein construct comprising an Fc domain;
a buffer selected from phosphate and tris(hydroxymethyl)aminomethane (TRIS);
one or more neutral amino acids e.g. selected from proline and glycine; and
an uncharged tonicity modifier e.g. sucrose;
   wherein the buffers are present in the composition at a total concentration of 0.1-5 mM; and
   wherein the total ionic strength of the composition excluding the contribution of the engineered protein construct is less than 20 mM.

In one embodiment, the invention provides an aqueous solution composition of pH in the range. 5.0 to 8.0 e.g. 5.0 to 7.5 e.g. 5.5 to 7.5. e.g. 6.0 to 7.5 comprising:
an engineered protein construct comprising an Fc domain;
a buffer selected from phosphate and citrate; and
an uncharged tonicity modifier e.g. sucrose;
   wherein the buffers are present in the composition at a total concentration of 0.1-5 mM; and
   wherein the total ionic strength of the composition excluding the contribution of the engineered protein construct is less than 20 mM.

In one embodiment, the invention provides an aqueous solution composition of pH in the range. 5.0 to 8.0 e.g. 5.0 to 7.5 e.g. 5.5 to 7.5. e.g. 6.0 to 7.5 comprising:
an engineered protein construct comprising an Fc domain;
a buffer selected from phosphate and citrate;
one or more neutral amino acids e.g. selected from proline and glycine; and
an uncharged tonicity modifier e.g. sucrose;
   wherein the buffers are present in the composition at a total concentration of 0.1-5 mM; and
   wherein the total ionic strength of the composition excluding the contribution of the engineered protein construct is less than 20 mM.

Suitably the composition of the invention remains as a clear solution following storage at 2-8° C. for extended period of time, such as at least 4 weeks, 8 weeks, 12 weeks, 12 months, 18 months or 24 months.

Suitably the composition of the invention remains as a clear solution following storage at 25° C. for extended period of time, such as at least 4 weeks, 8 weeks, 12 weeks, 12 months, 18 months or 24 months.

Suitably the composition of the invention remains as a clear solution following storage at 30° C. for extended period of time, such as at least 4 weeks, 8 weeks, 12 weeks, 12 months, 18 months or 24 months.

Suitably the composition of the invention remains as a clear solution following storage at 40° C. or 50° C. (i.e. temperatures suitable for accelerated stability trials) for a period of time, such as at least 1 day, 3 days, 1 week, 2 weeks or 4 weeks.

Suitably the composition of the invention has improved storage stability either at 2-8° C. or at increased temperature than in an equivalent composition that comprises higher concentration of the same buffer or buffers.

Suitably the composition of the invention has improved storage stability either at 2-8° C. or at increased temperature than in an equivalent composition that has a higher total ionic strength.

In one embodiment, the composition of the invention comprises no more than 5% total impurities, such as no more than 4%, such as no more than 3%, such as no more than 2% total impurities (by total weight of engineered protein construct in the composition, as measured by cation-exchange chromatography, size-exclusion chromatography or a similar suitable technique) following storage at 2-8° C. for at least 4 weeks, 8 weeks, 12 weeks, 12 months, 18 months or 24 months.

In one embodiment, the composition of the invention comprises no more than 5% total impurities, such as no more than 4%, such as no more than 3%, such as no more than 2% total impurities (by total weight of engineered protein construct in the composition, as measured by cation-exchange chromatography, size-exclusion chromatography or a similar suitable technique) following storage at 25° C. for at least 4 weeks, 8 weeks, 12 weeks, 12 months, 18 months or 24 months.

In one embodiment, the composition of the invention comprises no more than 5% total impurities, such as no more than 4%, such as no more than 3%, such as no more than 2% total impurities (by total weight of engineered protein construct in the composition, as measured by cation-exchange chromatography, size-exclusion chromatography or a similar suitable technique) following storage at 30° C. for at least 4 weeks, 8 weeks, 12 weeks, 12 months, 18 months or 24 months.

In one embodiment, the composition of the invention comprises no more than 5% total impurities, such as no more than 4%, such as no more than 3%, such as no more than 2% total impurities (by total weight of engineered protein construct in the composition, as measured by cation-exchange chromatography, size-exclusion chromatography or a similar suitable technique) following storage at 40° C. for at least 1 day, 3 days, 1 week, 2 weeks or 4 weeks.

In one embodiment, the composition of the invention comprises lower level of impurities (as measured by cation-exchange chromatography, size-exclusion chromatography or a similar suitable technique) than a commercially available composition comprising the same pharmaceutical ingredient (as measured the same technique(s)) following storage at 2-8° C. for at least 4 weeks, 8 weeks, 12 weeks, 12 months, 18 months or 24 months.

In one embodiment, the composition of the invention comprises lower level of impurities (as measured by cation-exchange chromatography, size-exclusion chromatography or a similar suitable technique) than a commercially available composition comprising the same pharmaceutical ingredient (as measured by the same technique(s)) following storage at 25° C. for at least 4 weeks, 8 weeks, 12 weeks, 12 months, 18 months or 24 months.

In one embodiment, the composition of the invention comprises lower level of impurities (as measured by cation-exchange chromatography, size-exclusion chromatography or a similar suitable technique) than a commercially available composition comprising the same pharmaceutical ingredient (as measured by the same technique(s)) following storage at 30° C. for at least 4 weeks, 8 weeks, 12 weeks, 12 months, 18 months or 24 months.

In one embodiment, the composition of the invention comprises lower level of impurities (as measured by cation-exchange chromatography, size-exclusion chromatography or a similar suitable technique) than a commercially available composition comprising the same pharmaceutical ingredient (as measured by the same technique(s)) following storage at 40° C. or 50° C. for at least 1 day, 3 days, 1 week, 2 weeks or 4 weeks.

In an embodiment, the composition of the invention is a composition for use in therapy. In an embodiment, the composition of the invention is a pharmaceutical composition. Compositions e.g. those intended for intravenous administration may be prepared as concentrates for dilution prior to administration.

All embodiments described above with respect to the aqueous solution composition apply equally to methods and uses of the invention.

There is also provided a container, for example made of plastics or glass, containing one dose or a plurality of doses of the composition as described herein. The container can be for example, a vial, a pre-filled syringe, a pre-filled infusion bag, or a cartridge designed to be a replaceable item for use with an injection device.

The compositions of the invention may suitably be packaged for injection, especially intravenous infusion, intravenous injection, subcutaneous injection or intramuscular injection.

An aspect of the invention is an injection or infusion device, particularly a device adapted for subcutaneous or intramuscular injection or infusion, for single or multiple use comprising a container containing one dose or a plurality of doses of the composition of the invention together with an injection needle. In an embodiment, the container is a replaceable cartridge which contains a plurality of doses. In one embodiment, the injection device is in the form of a pen. In one embodiment, the injection device is in the form of a pre-filled syringe. In one embodiment, the injection or infusion device is in the form of a pump or another wearable injection or infusion device.

Compositions according to the invention are expected to have good physical and chemical stability as described herein.

### EXAMPLES

### General Methods

### Methods of Assessing Stability of a Protein Construct

### (a) Visual Assessment

Visible particles are suitably detected using the 2.9.20. European Pharmacopoeia Monograph (Particulate Contamination: Visible Particles). The apparatus required consists of a viewing station comprising:
a matt black panel of appropriate size held in a vertical position
a non-glare white panel of appropriate size held in a vertical position next to the black panel
an adjustable lamp holder fitted with a suitable, shaded, white-light source and with a suitable light diffuser (a viewing illuminator containing two 13 W fluorescent tubes, each 525 mm in length, is suitable). The intensity of illumination at the viewing point is maintained between 2000 lux and 3750 lux.

Any adherent labels are removed from the container and the outside washed and dried. The container is gently swirled or inverted, ensuring that air bubbles are not introduced, and observed for about 5 s in front of the white panel. The procedure is repeated in front of the black panel. The presence of any particles is recorded.

The visual scores are ranked as follows:
Visual score A: Clear solution, virtually free of particles, <10 particles
Visual score B: Particles only visible under lamp
Visual score C: Significant visible change in appearance under normal laboratory conditions Whilst the particles in samples with visual scores C are clearly detectable on casual visual assessment under normal light, samples with visual score A and B generally appear as clear solutions on the same assessment. Samples with visual scores A and B are considered to be “Pass”; samples with visual score C are considered to be “Fail”.

### (b) Size Exclusion Chromatography (SEC)

The amount of high molecular weight species is measured using a 300×7.8 mm TSK Gel G3000 SWXL (or equivalent) size-exclusion column with a guard column. The mobile phase is sodium phosphate buffer pH 6.75, with a flow rate of 1 ml/min, injection volume of 20 μl and detected at 280 nm. The results are expressed as % high molecular species (HMWS), i.e. sum of all peak areas corresponding to aggregated protein over the sum of all protein-related peaks on the chromatogram. A small time-point to time-point variability can be observed in terms of absolute values of % Area (Monomer, HMWS and low molecular species (LMWS)), for example due to repeated size-exclusion column use. However, within a given time-point the samples are tested using the column in the same condition, so the values generated within the time-point represent a very good indication of the relative stability of the protein in the aqueous solutions tested.

### (c) Cation-Exchange Chromatography (CEX)

The amount of related species is measured using a Protein-Pak Hi Res SP column. Mobile phase A is 20 mM sodium phosphate (pH 6.5); mobile phase B is 20 mM sodium phosphate+0.5 M NaCl (pH 6.0). The following gradient elution is used: 0 min—100% A, 4 min—80% A, 10 min—55% A, 12 min—0% A. Flow rate of 1.0 ml/min; injection volume is 3 μl, with UV detection at 214 nm. The results are expressed as % main peak (i.e. native protein), % acidic species and % basic species. % Related species=% acidic species+% basic species.

### Example 1—Example Formulations

The following example formulations may be prepared:

### Example A

| | |
|---|---|
| Fc-fusion protein* | 50 mg/ml |
| Sodium phosphate | 1 mM |
| Sucrose | 300 mM |
| Water for injection | qs |

pH adjusted to 7.0 using either hydrochloric acid or sodium hydroxide

### Example B

| | |
|---|---|
| Fc-fusion protein* | 50 mg/ml |
| Sodium phosphate | 3 mM |
| Sucrose | 300 mM |
| Water for injection | qs |

pH adjusted to 7.0 using either hydrochloric acid or sodium hydroxide

### Example C

| | |
|---|---|
| Fc-fusion protein* | 50 mg/ml |
| TRIS | 1 mM |
| Sucrose | 300 mM |
| Water for injection | qs |

pH adjusted to 7.0 using either hydrochloric acid or sodium hydroxide

### Example D

| | |
|---|---|
| Fc-fusion protein* | 50 mg/ml |
| TRIS | 3 mM |
| Sucrose | 300 mM |
| Water for injection | qs |

pH adjusted to 7.0 using either hydrochloric acid or sodium hydroxide

### Example E

| | |
|---|---|
| Fc-fusion protein* | 50 mg/ml |
| Sodium phosphate | 1 mM |
| Sucrose | 300 mM |
| Proline | 100 mM |
| Water for injection | qs |

pH adjusted to 7.0 using either hydrochloric acid or sodium hydroxide

### Example F

| | | |
|---|---|---|
| Fc-fusion protein* | 50 | mg/ml |
| TRIS | 1 | mM |
| Sucrose | 300 | mM |
| Proline | 100 | mM |

| | |
|---|---|
| Water for injection | qs |

pH adjusted to 7.0 using either hydrochloric acid or sodium hydroxide

### Example G

| | | |
|---|---|---|
| Fc-fusion protein* | 50 | mg/ml |
| Sodium phosphate | 1 | mM |
| Sucrose | 300 | mM |
| Glycine | 100 | mM |

| | |
|---|---|
| Water for injection | qs |

pH adjusted to 7.0 using either hydrochloric acid or sodium hydroxide

### Example H

| | | |
|---|---|---|
| Fc-fusion protein* | 50 | mg/ml |
| TRIS | 1 | mM |
| Sucrose | 300 | mM |
| Glycine | 100 | mM |

| | |
|---|---|
| Water for injection | qs |

pH adjusted to 7.0 using either hydrochloric acid or sodium hydroxide

*Fc-fusion protein is (1) etanercept, (2) aflibercept or (3) dulaglutide.

### Examples AA-HH

The same formulations as Examples A-H, are prepared except that an engineered protein construct comprising an Fc domain which is a bispecific antibody instead of a Fc-fusion protein is used.

The stability of the formulations is determined using a visual assessment, SEC and CEX (see General Methods) following incubation at 40° C. for 2, 4 and 8 weeks.

The stability of the formulations is determined using a visual assessment, SEC and CEX (see General Methods) following incubation at 25° C. for 2, 4, 8 and 12 weeks.

The stability of the formulations is determined using a visual assessment, SEC and CEX (see General Methods) following incubation at 2-8° C. for 2, 4, 8 and 12 weeks.

### Example 2—Effect of Buffer Concentration and Charge of the Tonicity Modifier on Stability of Dulaglutide at 40° C. And 50° C.

The effect of buffer concentration and charge of the tonicity modifier on stability of dulaglutide (1 mg/ml) was investigated. Citrate buffer and phosphate buffer were tested. Sodium chloride (150 mM) was used as a charged tonicity modifier and sucrose (250 mM) was used as an uncharged tonicity modifier. All formulations tested were adjusted to pH 6.5. In addition, the formulation of the marketed dulaglutide product (Trulicity) was used as a Control formulation for comparison. Table 1 summarizes the formulations tested. All formulations were stressed at 40° C. and 50° C. for 4 weeks. Stability of dulaglutide was followed by monitoring the rate of high molecular weight species formation using SEC.

TABLE 1
Formulations of dulaglutide tested. All formulations contained dulaglutide
(1 mg/ml) and were adjusted to pH 6.5.

| | Citric | Sodium | Sodium | | | |
|---|---|---|---|---|---|---|
| | acid | phosphate | chloride | Sucrose | Mannitol | Tween 80 |
| | (mM) | (mM) | (mM) | (mM) | (mM) | (mg/ml) |
| Formulation 2-1 | 1 | | 150 | 0 | | |
| Formulation 2-2 | 5 | | 150 | 0 | | |
| Formulation 2-3 | 10 | | 150 | 0 | | |
| Formulation 2-4 | 50 | | 150 | 0 | | |
| Formulation 2-5 | 1 | | 0 | 250 | | |
| Formulation 2-6 | 3 | | 0 | 250 | | |
| Formulation 2-7 | 5 | | 0 | 250 | | |
| Formulation 2-8 | 10 | | 0 | 250 | | |
| Formulation 2-9 | 50 | | 0 | 250 | | |
| Formulation 2-10 | | 1 | 150 | 0 | | |
| Formulation 2-11 | | 5 | 150 | 0 | | |
| Formulation 2-12 | | 10 | 150 | 0 | | |
| Formulation 2-13 | | 50 | 150 | 0 | | |
| Formulation 2-14 | | 1 | 0 | 250 | | |
| Formulation 2-15 | | 3 | 0 | 250 | | |
| Formulation 2-16 | | 5 | 0 | 250 | | |
| Formulation 2-17 | | 10 | 0 | 250 | | |
| Formulation 2-18 | | 50 | 0 | 250 | | |
| Formulation 2-19 | 10* | | | | 250 | 0.067 |
| (Control formulation) | | | | | | |

*prepared by mixing sodium citrate (9.3 mM) with citric acid (0.7 mM)

All formulations tested passed the visual test (Visual score A) following storage at both temperatures. The rate of HMWS formation in formulations 2-1 to 2-19 following storage at 50° C. and 40° C. is shown in Table 2. The rate of HMWS formation was lowest in formulations comprising a very low buffer concentration and an uncharged tonicity modifier. In the presence of sucrose, no detectable increase of HMWS was observed at 40° C. in compositions comprising up to 5 mM citrate buffer. The rate of HMWS formation increased at higher concentrations of citrate buffer. Similarly, under a more accelerated stress at 50° C., the rate of HMWS formation was proportional to the concentration of citrate buffer, being very low at buffer concentrations of 5 mM or less. Similar observations were made using phosphate buffer. In contrast, HMWS formation was observed in the presence of sodium chloride at all buffer concentrations.

TABLE 2
Stability of dulaglutide (1 mg/ml) at 40° C. and 50° C. in
formulations 2-1 to 2-19 assessed by SEC.

| | Increase in % HMWS following | |
|---|---|---|
| | incubation at indicated temperature | |
| | 50° C. (4 weeks) | 40° C. (4 weeks) |
| Formulation 2-1 | 7.97 | 0.80 |
| Formulation 2-2 | 6.73 | 0.68 |
| Formulation 2-3 | 8.33 | 1.03 |
| Formulation 2-4 | 11.18 | 1.33 |
| Formulation 2-5 | 0.06 | 0 |
| Formulation 2-6 | 0.05 | 0 |
| Formulation 2-7 | 2.17 | 0 |
| Formulation 2-8 | 5.2 | 0.41 |
| Formulation 2-9 | 11.92 | 1.74 |
| Formulation 2-10 | 8.9 | 0.64 |
| Formulation 2-11 | 12.01 | 0.60 |
| Formulation 2-12 | 6.86 | 0.68 |
| Formulation 2-13 | 10.81 | 0.90 |
| Formulation 2-14 | Not available* | Not available* |
| Formulation 2-15 | 0.77 | 0 |
| Formulation 2-16 | 3.26 | 0 |
| Formulation 2-17 | 4.56 | 0.23 |
| Formulation 2-18 | 17.44 | 1.74 |
| Formulation 2-19 | 4.24 | 0.32 |
| (Control formulation) | | |

*unfortunately the testing of this formulation failed for reasons unrelated to the formulation itself

### Example 3—Effect of Buffer Concentration and Charge of the Tonicity Modifier on Stability of Abatacept at 50° C.

The effect of buffer concentration and charge of the tonicity modifier on stability of abatacept (4.25 mg/ml) was investigated. Citrate buffer and phosphate buffer were tested. Sodium chloride (150 mM) was used as a charged tonicity modifier and sucrose (250 mM) was used as an uncharged tonicity modifier. All formulations tested were adjusted to pH 6.8. In addition, the formulation of the marketed abatacept product (Orencia) was used as a Control formulation for comparison. Table 3 summarizes the formulations tested. All formulations were stressed at 50° C. for 2 weeks. Stability of abatacept was followed by monitoring the rate of high molecular weight species formation using SEC.

TABLE 3
Formulations of abatacept tested. All formulations contained
abatacept (4.25 mg/ml) and were adjusted to pH 6.8.

| | Citric | Sodium | Sodium | | |
|---|---|---|---|---|---|
| | acid | phosphate | chloride | Sucrose | Maltose |
| | (mM) | (mM) | (mM) | (mM) | (mM) |
| Formulation 3-1 | 1 | | 150 | 0 | |
| Formulation 3-2 | 5 | | 150 | 0 | |
| Formulation 3-3 | 10 | | 150 | 0 | |
| Formulation 3-4 | 50 | | 150 | 0 | |
| Formulation 3-5 | 1 | | 0 | 250 | |
| Formulation 3-6 | 3 | | 0 | 250 | |
| Formulation 3-7 | 5 | | 0 | 250 | |
| Formulation 3-8 | 10 | | 0 | 250 | |
| Formulation 3-9 | 50 | | 0 | 250 | |
| Formulation 3-10 | | 1 | 150 | 0 | |
| Formulation 3-11 | | 5 | 150 | 0 | |
| Formulation 3-12 | | 10 | 150 | 0 | |
| Formulation 3-13 | | 50 | 150 | 0 | |
| Formulation 3-14 | | 1 | 0 | 250 | |
| Formulation 3-15 | | 3 | 0 | 250 | |
| Formulation 3-16 | | 5 | 0 | 250 | |
| Formulation 3-17 | | 10 | 0 | 250 | |
| Formulation 3-18 | | 50 | 0 | 250 | |
| Formulation 3-19 | | 12.5 | 25 | | 138.8 |
| (Control formulation) | | | | | |

All formulations tested passed the visual test (Visual score A) following storage at 50° C. The rate of HMWS formation in formulations 3-1 to 3-19 following storage at 50° C. is shown in Table 4. The rate of HMWS formation was lowest in formulations comprising a very low buffer concentration and an uncharged tonicity modifier. The rate of HMWS formation increased with increasing buffer concentration, both in the case of citrate and in the case of phosphate buffer. In contrast, HMWS formation was very high in the presence of sodium chloride regardless of buffer concentration.

TABLE 4
Stability of abatacept (4.25 mg/ml) at 50° C. in
formulations 3-1 to 3-19 assessed by SEC.

| | Increase in % HMWS |
|---|---|
| | following incubation |
| | at 50° C. for 2 weeks |
| Formulation 3-1 | 84.33 |
| Formulation 3-2 | 85 |
| Formulation 3-3 | 83.39 |
| Formulation 3-4 | 85.49 |
| Formulation 3-5 | 19.35 |
| Formulation 3-6 | 22.92 |
| Formulation 3-7 | 34.84 |
| Formulation 3-8 | 54.39 |
| Formulation 3-9 | 76.36 |
| Formulation 3-10 | 83.8 |
| Formulation 3-11 | 82.56 |
| Formulation 3-12 | 82.78 |
| Formulation 3-13 | 83.96 |
| Formulation 3-14 | 24.81 |
| Formulation 3-15 | 31.36 |
| Formulation 3-16 | 38.80 |
| Formulation 3-17 | 48.20 |
| Formulation 3-18 | 72.71 |
| Formulation 3-19 | 70.58 |
| (Control formulation) | |

### Example 4—Effect of Buffer Concentration and Charge of the Tonicity Modifier on Stability of Abatacept at 40° C.

The effect of citrate buffer concentration and charge of the tonicity modifier on stability of abatacept (4.25 mg/ml) was investigated. Sodium chloride (150 mM) was used as a charged tonicity modifier and sucrose (250 mM) was used as an uncharged tonicity modifier. All formulations tested were adjusted to pH 6.8. Table 5 summarizes the formulations tested. All formulations were stressed at 40° C. for 2 weeks. Stability of abatacept was followed by monitoring the rate of high molecular weight species formation using SEC.

TABLE 5
Formulations of abatacept tested. All formulations contained
abatacept (4.25 mg/ml) and were adjusted to pH 6.8.

| | Citric | Sodium | Sodium | | |
|---|---|---|---|---|---|
| | acid | phosphate | chloride | Sucrose | Maltose |
| | (mM) | (mM) | (mM) | (mM) | (mM) |
| Formulation 4-1 | 1 | | 150 | 0 | |
| Formulation 4-2 | 5 | | 150 | 0 | |
| Formulation 4-3 | 10 | | 150 | 0 | |
| Formulation 4-4 | 50 | | 150 | 0 | |
| Formulation 4-5 | 1 | | 0 | 250 | |
| Formulation 4-6 | 3 | | 0 | 250 | |
| Formulation 4-7 | 5 | | 0 | 250 | |
| Formulation 4-8 | 10 | | 0 | 250 | |
| Formulation 4-9 | 50 | | 0 | 250 | |

All formulations tested passed the visual test (Visual score A) following storage at 40° C. The rate of HMWS formation in formulations 4-1 to 4-9 following storage at 40° C. is shown in Table 6. The rate of HMWS formation was lowest in composition comprising a very low citrate buffer concentration and an uncharged tonicity modifier. The rate of HMWS formation increased with increasing citrate buffer concentration. In contrast, HMWS formation was very high in the presence of sodium chloride regardless of buffer concentration.

TABLE 6
Stability of abatacept (4.25 mg/ml) at 40° C. in
formulations 4-1 to 4-9 assessed by SEC.

| | Increase in % HMWS |
|---|---|
| | following incubation |
| | at 40° C. for 2 weeks |
| Formulation 4-1 | 16.55 |
| Formulation 4-2 | 17.69 |
| Formulation 4-3 | 17.21 |
| Formulation 4-4 | 19.64 |
| Formulation 4-5 | 2.61 |
| Formulation 4-6 | 3.03 |
| Formulation 4-7 | 3.80 |
| Formulation 4-8 | 4.72 |
| Formulation 4-9 | 7.42 |

### Example 5—Effect of Proline on Stability of Abatacept at 50° C. In the Presence of 1 mM Buffer and Uncharged Tonicity Modifier

The effect of proline (50 mM) on stability of abatacept (4.25 mg/ml) was investigated in the presence of 1 mM buffer and uncharged tonicity modifier. Sucrose (250 mM) was used as an uncharged tonicity modifier. All formulations tested were adjusted to pH 6.8. Table 7 summarizes the formulations tested. All formulations were stressed at 50° C. for 2 weeks. Stability of abatacept was followed by monitoring the rate of high molecular weight species formation using SEC.

TABLE 7
Formulations of abatacept tested. All formulations contained
abatacept (4.25 mg/ml) and were adjusted to pH 6.8.

| | Citric | Sodium | | |
|---|---|---|---|---|
| | acid | phosphate | Sucrose | Proline |
| | (mM) | (mM) | (mM) | (mM) |
| Formulation 5-1 | 1 | | 250 | |
| Formulation 5-2 | 1 | | 250 | 50 |
| Formulation 5-3 | | 1 | 250 | |
| Formulation 5-4 | | 1 | 250 | 50 |

All formulations tested passed the visual test (Visual score A) following storage at 50° C. The rate of HMWS formation in formulations 5-1 to 5-4 following storage at 50° C. is shown in Table 8. The presence of proline (50 mM) in compositions comprising 1 mM buffer and sucrose (250 mM) resulted in a lower rate of HMWS formation.

TABLE 8
Stability of abatacept (4.25 mg/ml) at 50° C. in
formulations 5-1 to 5-4 assessed by SEC.

| | Increase in % HMWS |
|---|---|
| | following incubation |
| | at 50° C. for 2 weeks |
| Formulation 5-1 | 19.35 |
| Formulation 5-2 | 17.43 |
| Formulation 5-3 | 24.81 |
| Formulation 5-4 | 22.58 |

### Example 6—Effect of Proline and Glycine on Stability of Abatacept at 40° C. In the Presence of 1 mM Buffer and Uncharged Tonicity Modifier

The effect of proline (50 mM) and glycine (50 mM) on stability of abatacept (4.25 mg/ml) was investigated in the presence of 1 mM buffer and uncharged tonicity modifier. Citrate buffer and phosphate buffer were tested. Sucrose (250 mM) was used as an uncharged tonicity modifier. All formulations tested were adjusted to pH 6.8. Table 9 summarizes the formulations tested. All formulations were stressed at 40° C. for 2 weeks. Stability of abatacept was followed by monitoring the rate of high molecular weight species formation using SEC.

TABLE 9
Formulations of abatacept tested. All formulations contained
abatacept (4.25 mg/ml) and were adjusted to pH 6.8.

| | Citric | Sodium | | | |
|---|---|---|---|---|---|
| | acid | phosphate | Sucrose | Proline | Glycine |
| | (mM) | (mM) | (mM) | (mM) | (mM) |
| Formulation 6-1 | 1 | | 250 | | |
| Formulation 6-2 | 1 | | 250 | 50 | |
| Formulation 6-3 | 1 | | 250 | | 50 |
| Formulation 6-4 | | 1 | 250 | | |
| Formulation 6-5 | | 1 | 250 | 50 | |
| Formulation 6-6 | | 1 | 250 | | 50 |

All formulations tested passed the visual test (Visual score A) following storage at 40° C. The rate of HMWS formation in formulations 6-1 to 6-6 following storage at 40° C. is shown in Table 10. The presence of proline (50 mM) and glycine (50 mM) in compositions comprising 1 mM buffer and sucrose (250 mM) resulted in a lower rate of HMWS formation.

TABLE 10
Stability of abatacept (4.25 mg/ml) at 40° C. in
formulations 6-1 to 6-6 assessed by SEC.

| | Increase in % HMWS |
|---|---|
| | following incubation |
| | at 40° C. for 2 weeks |
| Formulation 6-1 | 2.61 |
| Formulation 6-2 | 1.64 |
| Formulation 6-3 | 1.81 |
| Formulation 6-4 | 5.37 |
| Formulation 6-5 | 3.69 |
| Formulation 6-6 | 4.82 |

### Comparative Example 7—Effect of Buffer Concentration, Charge of Tonicity Modifier and a Neutral Amino Acid on Stability of an Immunoglobulin G1 (IgG1) at 30° C.

The effect of buffer concentration and charge of the tonicity modifier on stability of an IgG1 (100 mg/ml) was investigated. Citrate buffer was tested. Sodium chloride (150 mM) was used as a charged tonicity modifier and glycerol (300 mM) was used as an uncharged tonicity modifier. The effect of proline (50 mM and 200 mM) on stability of the IgG1 was also investigated in the presence of 1 mM buffer and both modifiers. All formulations tested contained polysorbate 80 (0.2 mg/ml) and were adjusted to pH 6.0. Table 11 summarizes the formulations tested. All formulations were stressed at 30° C. for 8 weeks. Stability of the IgG1 was followed by monitoring the rate of high molecular weight species formation using SEC.

TABLE 11
Formulations of IgG1 tested. All formulations
contained IqG1 (100 mq/ml) and polysorbate 80
(0.2 mq/ml) and were adjusted to pH 6.0.

| | Citric | Sodium | | | |
|---|---|---|---|---|---|
| | acid | chloride | Glycerol | Proline | Glycine |
| | (mM) | (mM) | (mM) | (mM) | (mM) |
| Formulation 7-1 | 1 | 150 | | | |
| Formulation 7-2 | 5 | 150 | | | |
| Formulation 7-3 | 20 | 150 | | | |
| Formulation 7-4 | 1 | | 300 | | |
| Formulation 7-5 | 5 | | 300 | | |
| Formulation 7-6 | 20 | | 300 | | |
| Formulation 7-7 | 1 | | 300 | 50 | |
| Formulation 7-8 | | | 300 | | 50 |

All formulations tested passed the visual test (Visual score A) following storage at 30° C. The rate of HMWS formation in formulations 7-1 to 7-8 following storage at 30° C. is shown in Table 12. The rate of HMWS formation increased with increasing buffer concentration both in the presence of sodium chloride and in the presence of glycerol. The rate of HMWS formation at a given buffer concentration appeared not to depend on the nature of the tonicity modifier. The presence of a neutral amino acid (proline or glycine) resulted in a very slight increase in the rate of HMWS formation.

TABLE 12
Stability of IgG1 (100 mg/ml) at 30° C. in
formulations 7-1 to 7-8 assessed by SEC.

| | Increase in % HMWS following |
|---|---|
| | incubation at 30° C. for 8 weeks |
| Formulation 7-1 | 6.28 |
| Formulation 7-2 | 5.15 |
| Formulation 7-3 | 4.41 |
| Formulation 7-4 | 6.58 |
| Formulation 7-5 | 5.48 |

| | |
|---|---|
| Formulation 7-6 | 4.27 |
| Formulation 7-7 | 6.67 |
| Formulation 7-8 | 6.68 |

### SUMMARY OF THE EXAMPLES

The data of Examples 2-6 shows that formulations of an engineered protein construct containing an Fc domain are stable when containing buffer up to 5 mM and at low ionic strength (such as less than 20 mM, excluding the contribution of the engineered protein construct). Higher buffer concentrations and higher ionic strength were destabilising. The presence of a neutral amino acid was further stabilising. Surprisingly this is opposite to the behaviour exhibited by the tested 4-chain antibody (type IgG1). The data of Comparative Example 7 shows that this antibody was more stable at higher buffer concentrations and higher ionic strength. The presence of a neutral amino acid was further destabilising.

Without being limited by theory, the inventors believe that compared with natural proteins or recombinant proteins based on natural structural templates (e.g. immunoglobulins) the expression of novel engineered protein constructs comprising an Fc domain results in a greater abundance of hydrophobic patches at the protein surface as well as a greater exposure of sites of instability, such as regions prone to hydrolytic cleavage. This in turn leads to greater propensity to aggregation and structural degradation. The present invention combines formulation features that, without being limited by theory, are believed to work in concert to screen the unnatural hydrophobic patches as well as minimising the rate of proton exchange at the unnaturally exposed sites of instability, resulting in substantial stability improvement of engineered protein constructs comprising an Fc domain.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents, patent applications and references mentioned throughout the specification of the present invention are herein incorporated in their entirety by reference.

The invention embraces all combinations of preferred and more preferred groups and suitable and more suitable groups and embodiments or groups recited above.

1. An aqueous solution composition of pH in the range 4.0-8.5 comprising:
an engineered protein construct comprising an Fc domain;
optionally one or more buffers being substances having at least one ionisable group with a pKₐ in the range 3.0 to 9.5 and which pKₐ is within 2 pH units of the pH of the composition;
optionally one or more neutral amino acids; and
an uncharged tonicity modifier;
wherein the buffers are present in the composition at a total concentration of 0-5 mM; and
wherein the total ionic strength of the composition excluding the contribution of the engineered protein construct is less than 20 mM.

2. An aqueous solution composition according to claim 1 wherein buffers are present at a total concentration of 0.1-5 mM, such as 0.1-4 mM, 0.1-3 mM, 0.1-2 mM or 0.1-1 mM.

3. An aqueous solution composition according to claim 1 wherein buffers are present at a total concentration of 1-5 mM, such as 1˜4 mM or 1-3 mM.

4. An aqueous solution composition according to claim 1, wherein the total concentration of buffers in the composition is <4.5 mM, such as <4 mM, <3 mM, <2 mM, <1 mM, <0.5 mM, <0.4 mM, <0.3 mM, <0.2 mM or <0.1 mM.

5. An aqueous solution composition according to claim 1 which is substantially free of buffers.

6. An aqueous solution composition according to any one of claims 1, 2, 3 and 4, wherein the buffer comprises ionisable groups with pKₐ within 1 unit of the pH of the composition.

7. An aqueous solution composition according to any one of claims 1 to 4 and 6, wherein the buffer or buffers is/are selected from the group consisting of citrate, histidine, maleate, sulphite, glyoxylate, aspartame, glucuronate, aspartate, glutamate, tartrate, gluconate, lactate, glycolic acid, adenine, succinate, ascorbate, benzoate, phenylacetate, gallate, cytosine, p-aminobenzoic acid, sorbate, acetate, propionate, alginate, urate, 2-(N-morpholino) ethanesulphonic acid, bicarbonate, bis(2-hydroxyethyl) iminotris(hydroxymethyl) methane, N-(2-acetamido)-2-iminodiacetic acid, 2-[(2-amino-2-oxoethyl)amino]ethanesulphonic acid, piperazine, N,N′-bis(2-ethanesulphonic acid), phosphate, N,N-bis(2-hydroxyethyl)-2-aminoethanesulphonic acid, 3-[N,N-bis(2-hydroxyethyl)amino]-2-hydroxypropanesulphonic acid, triethanolamine, piperazine-N,N′-bis(2-hydroxypropanesulphonic acid), tris(hydroxymethyl)aminomethane, N-tris(hydroxymethyl)glycine and N-tris(hydroxymethyl)methyl-3-aminopropanesulphonic acid, and salts thereof, and combinations thereof.

8. An aqueous solution composition according to claim 7, wherein the buffer is selected from the group consisting of citrate, histidine, maleate, tartrate, lactate, benzoate, acetate, bicarbonate, phosphate and tris(hydroxymethyl)aminomethane, for example, selected from citrate and phosphate.

9. An aqueous solution composition according to any one of claims 1 to 8 comprising a polyol as an uncharged tonicity modifier.

10. An aqueous solution composition according to any one of claims 1 to 8 comprising an uncharged tonicity modifier selected from glycerol, 1,2-propanediol, mannitol, sorbitol, sucrose, trehalose, PEG300 and PEG400 and in particular selected from glycerol, mannitol, 1,2-propanediol and sucrose.

11. An aqueous solution composition according to any one of claims 1 to 10 wherein the concentration of the uncharged tonicity modifier is 50-1000 mM, such as 200-500 mM, or about 300 mM.

12. An aqueous solution composition according to any one of claims 1 to 11 comprising one or more neutral amino acids selected from glycine, methionine, proline, alanine, valine, leucine, isoleucine, phenylalanine, tyrosine, tryptophan, serine, threonine, asparagine, glutamine.

13. An aqueous solution composition according to claim 12 comprising one or more neutral amino acids selected from glycine, methionine, proline and alanine.

14. An aqueous solution composition according to claim 13 comprising proline as a neutral amino acid.

15. An aqueous solution composition according to claim 13 comprising glycine as a neutral amino acid.

16. An aqueous solution composition according to any one of claims 1 to 15 wherein the total concentration of the one or more neutral amino acids in the formulation is 20 to 200 mM e.g. 50-150 mM.

17. An aqueous solution composition according to any one of claims 1 to 16 wherein the total ionic strength of the composition excluding the contribution of the engineered protein construct is less than 10 mM.

18. An aqueous solution composition according to any one of claims 1 to 17 wherein the pH is between 4.0 and 7.5, such as between 5.0 and 7.5 e.g. between 5.5 and 7.5. e.g. between 6.0 and 7.5, such as between 7.0 and 7.5.

19. An aqueous solution composition according to any one of claims 1 to 18, wherein the engineered protein construct is a fusion of an Fc domain with a heterologous polypeptide.

20. An aqueous solution composition according to claim 19, wherein the heterologous polypeptide is selected from cytokines, growth factors, blood clotting factors, enzymes, receptor proteins, GLP-1 agonists and functional fragments and domains thereof.

21. An aqueous solution composition according to claim 20, wherein the engineered protein construct is selected from etanercept, abatacept, belatacept, aflibercept, rilonacept, romiplostim, eloctate, luspatercept, dulaglutide and alprolix.

22. An aqueous solution composition according to claim 20, wherein the heterologous polypeptide is a protease inhibitor.

23. An aqueous solution composition according to any one of claims 1 to 18, wherein the engineered protein construct is a bispecific antibody in the format of a 4-chain antibody having two different variable binding regions.

24. An aqueous solution composition according to any one of claims 1 to 18, wherein the engineered protein construct is a bispecific antibody in the format of a 2-chain antibody having two different variable binding regions.

25. An aqueous solution composition according to any one of claims 1 to 24, wherein the protein is present at a concentration of 1 to 400 mg/ml e.g. 10-200 mg/ml e.g. 20-100 mg/ml.

26. An aqueous solution composition according to any one of claims 1 to 25, which comprises a non-ionic surfactant.

27. An aqueous solution composition according to claim 26, wherein the non-ionic surfactant is selected from the group consisting of an alkyl glycoside, a polysorbate, an alkyl ether of polyethylene glycol, a block copolymer of polyethylene glycol and polypropylene glycol, and an alkylphenyl ether of polyethylene glycol.

28. An aqueous solution composition according to claim 27, wherein the non-ionic surfactant is a polysorbate such as polysorbate 20 or polysorbate 80.

29. An aqueous solution composition according to any one of claims 26 to 28, wherein the non-ionic surfactant is present at a concentration of 10-2000 μg/ml, such as 50-1000 μg/ml, 100-500 μg/ml or about 200 μg/ml.

30. An aqueous solution composition according to any one of claims 1 to 29, which comprises a preservative such as a phenolic or benzylic preservative.

31. An aqueous solution composition according to claim 30, wherein the phenolic or benzylic preservative is selected from the group consisting of phenol, m-cresol, chlorocresol, benzyl alcohol, propyl paraben and methyl paraben.

32. An aqueous solution composition according to claim 30 or claim 31, wherein the preservative is present at a concentration of 10-100 mM, such as 20-80 mM or 25-50 mM.

33. An aqueous solution composition according to any one of claims 1 to 32, which is a composition for use in therapy.

34. An aqueous solution composition according to any one of claims 1 to 33, which is a pharmaceutical composition.
